(19) United States
(12) Patent Application Publication
(10) Pub. No.: US 2002/0058864 A1
(43) Pub. Date: May 16, 2002

(54) Reduction of spectral site to site variation

(76) Inventors: James R. Mansfield, Boston, MA (US); Robert G. Messerschmidt, Albuquerque, NM (US); Solomon Marini, Lexington, MA (US); Pierre Trepagnier, Medford, MA (US)

Correspondence Address: HELLER EHRMAN WHITE & MCAULIFFE LLP, SUITE 300, 101 ORCHARD RIDGE DR., GAITHERSBURG, MD, 20878-1917, US

(21) Appl. No.: 09/987,192

(22) Filed: Nov. 13, 2001

Related U.S. Application Data
(60) Provisional application No. 60/247,002, filed on Nov. 13, 2000.

(51) Int. Cl. A61B 5/00

(52) U.S. Cl. 600/316; 600/317

(57) ABSTRACT

The invention relates to devices and methods that improve the quality of optic measurements from surfaces such as skin and biological materials. Three methods for reducing spectral site to site variation in fluorescence and/or reflectance signals obtained from a sample surface are: repeated measurements taken at identifiable location(s) determined by fiducial marks, repeat of measurements at different locations on the sample, and tensioning the sample surface during measurement to alleviate surface heterogeneity. Combinations of these methodologies provide best results, and are expected to improve the ability to measure blood glucose non-invasively.

### REFERENCE TO RELATED APPLICATIONS

This application claims priority to U.S. Provisional application No. 60/247,002, entitled “Reduction of Spectral Site to Site Variation” filed Nov. 13, 2000, the contents of which are incorporated by reference in their entirety.

### FIELD OF THE INVENTION

The invention relates to methods and devices for spectral optic measurements of skin and other surfaces.

### BACKGROUND

Skin fluorescence spectra measurements are useful for diagnosing various conditions of the skin and often are used in the cosmetics industry. Such measurements typically involve a fiber optic probe, which is pressed against the skin, a light source with an optional light filter or grating, and a detector. Commercially available instruments have been developed, such as the Skinskan, (Instruments S.A. Inc.) that incorporate these components to generate spectroscopic measurements. Combinations of simple (non-imaging) fluorescence and reflectance spectra have been used to diagnose conditions as described in U.S. Pat. Nos. 6,008,889 and 6,069,689 issued to Zeng et al. on May 28, 1999 and May 30, 2000 respectively.

Unfortunately, however, a spectra measurement often differs typically even when taken in the same general area of skin, and such instrument measurements are susceptible to error. Some of this error arises from exogenous factors such as pressure and temperature. Local differences in the skin make up a particularly large portion of this total variation error. Such local variation is termed “site-to-site variation.” Work by others in this field as reported in U.S. Pat. Nos. 6,008,889 and 6,069,689 do not address satisfactorily this variation. This limitation, in fact can be considered as hindering progress in the use of fluorescence for detecting the condition of a sample (such as skin) or detecting blood analytes.

The site-to-site variation arises from, among other things, 1) non-uniformity of skin pigmentation is (i.e. many local variations), non uniform thickness of the skin (containing many internal folds), various scattering properties and thicknesses of the stratum corneum and epidermis, which leads to differential absorptions, a non-homogeneous distribution of collagen, which contains fluorophores and which may itself be non-uniform and anisotropic, and the skin's non-uniform texture, which includes small hills and valleys in the surface.

Site-to-site measurement variation due to these factors complicates the use of skin fluorescence spectra for quantitation. The variation acts as a noise source and can mask small changes in the spectra. Such small changes may affect, for example, clinical judgements and other results. These problems are particularly limiting when the spectral data are used to monitor blood analytes such as glucose. Accordingly, an important goal in acquiring fluorescence spectra is to minimize such errors.

### SUMMARY OF THE INVENTION

The invention alleviates disadvantages with current strategies and designs for obtaining fluorescence spectra on tissue surfaces by providing methods and apparatus that reduce errors from repeated measurements and from spectral site to site variation.

One embodiment of the invention is a method of minimizing error in optic spectra from a sample comprising the steps of applying fixed fiducial points to the sample surface and referencing an optical probe to those fiducial points, so that the spectra are always taken in the same place. Another embodiment is a method of minimizing the variation of optic spectra from a sample comprising the steps of gathering a plurality of spectra at nearby points on the sample and combining the spectra so as to form a representative measurement. Yet another embodiment is a method of minimizing the variation of measured optic spectra from a flexible sample surface comprising tensioning the sample surface prior to or at the time of making a spectral measurement with an optical probe. Other embodiments will be appreciated by a reading of the specification and consideration of the referenced documents that provide further details for making and using the invention for a wide range of diagnostics.

### DESCRIPTION OF THE DRAWINGS

FIG. 1 shows a tensioning arrangement according to an embodiment of the invention.

FIG. 2 shows a fiber optic probe having four apertures according to an embodiment of the invention.

FIG. 3 shows a one piece mounting surface with multiple attachment points according to an embodiment of the invention.

### DESCRIPTION OF THE PREFERRED EMBODIMENTS

While exploring the limits of spectroscopic measurements from skin under the most demanding of applications, namely for the determination of blood glucose, the inventors made several discoveries. In a first discovery site-to-site variation was minimized by providing fiducial optical points. These points allow probe re-registration so that, among other things spectra can be taken more reproducibly from a sample. In a second discovery, site to site variation was controlled by taking spectra measurements at multiple skin sites and averaging the spectra over these skin sites. This latter approach minimizes variation by effectively sampling many sites simultaneously.

In a third discovery tensioning the skin slightly (typically 0.5%, but a wide range from 0.1% to 10% and even 0.01% to 50%) before taking the spectra was found to improve measurements. This tensioning can reduce site-to-site variation and also variation from multiple measurements from the same site. Without wishing to be bound by any one particular theory of the invention it is thought that tensioning of an elastic sample surface such as the skin of an animal or plant minimizes the effect of folds, hills, and valleys on the surface, and thereby reduces spectral variation. The sample surface tensioning may be carried out during measurement by placing one or more physical (mechanical) fiducial points on the skin with a spacing slightly smaller than that of the probe into which they will fit. Attaching the probe to the skin will thus slightly spread apart the fiducial points and tension (stretch) the skin. In yet another embodiment the skin is tensioned by a device that allows a probe to be placed multiple times at multiple positions on the tensioned portion.

Site to site variation may be decreased in these independent ways as outlined here but in some embodiments two or more of the methods are combined. For example, fiducial optical points may be combined with skin tensioning to control or even measure the degree of tensioning, to further improve the quality of assay result. The use of fiducial optical points with multiple sites allows further assay improvements by multiple measurements at multiple locations. Yet further combinations of the three features are possible as may be appreciated by a reading of the patent specification.

### Fluorescent and Reflectance Measurements with a Probe

Desirable embodiments of the invention utilize a probe to train fluorescence excitation light to a spot on the sample surface and to pick up fluorescence emission light from the surface. Other embodiments may use the same probe conformation to train light onto the spot and pick up reflected light. In many embodiments a sample is a biological tissue such as skin tissue. Skin measurements may be used according to a preferred embodiment of the invention to quantitate the level of glucose and/or other blood solutes. Skin measurements also can detect or monitor other substances such as aging pigments and other features associated with a skin disease such as for example, squamous cell carcinoma, seborrheic keratosis, spider angioma, actinic keratosis, compound nevus and psoriasis. Skin measurements further may be used to detect or quantitate conditions that lead to or result from diseases such as diabetes, other cancers such as cancers of the blood, liver disorders, vitamin deficiencies or excess, hemoglobin status, hematocrit and the like.

The invention may be used for a wide range of samples, including biological materials such as an internal organ during surgery, an excised tissue such as a suspected cancerous growth, a bodily fluid, a dried body fluid such as a blood specimen for forensic testing, a tongue, or web of skin. A biological sample is not limited to that from a human being but may be from another animal or another type of organism such as a tree. For example, a mutant tree that has been genetically modified to synthesize less lignin or with more efficient photosynthesis can be detected by florescence means because of the different spectral properties that result from the different lignin/cellulose contents and different chloroplast composition, respectively. In some embodiments, as a skilled artisan will readily appreciate, polarized filters may be used to detect for the rotation of plane polarized light, as may be used to detect or quantitate chiral materials, and particularly polymers that stack in a semi crystalline manner.

Generally, fluorescence spectra are generated with (1) an excitation light source, (2) a focusing mechanism or other mechanism for bringing or confining the excitation light onto the tissue surface and to gather emission light, and (3) a detector of fluorescence emission. The types of light sources, optical filters as needed, focusing mechanisms, detectors, data storage devices and the like are well known, as for example described in U.S. Pat. Nos. 6,008,889; 6,069,689; 5,786,893; 5,784,162; 5,778,016; 5,769,081; 5,753,511; 5,751,415; 5,738,101; 5,705,518; 5,701,901; 5,699,795; 5,697,373; 5,693,043; 5,687,730; 5,647,368; 5,615,673 and 5,601,087. These documents are incorporated by reference in their entireties. The descriptions in these documents of light sources, optical filtering, focusing mechanisms, detectors and methods of their use are most particularly incorporated by reference, as space limitations preclude repeating this detailed information.

In preferred embodiments two or more fluorescence spectra are compared with stored or calculated spectra data and other information corresponding to known or calibrated optical properties of test materials to generate a test result. The reference information may be used as calibrators for determining a relative nutritional quality, amount, quality, environmental exposure, genetic heritage, age, exposure to environmental variable(s) or toxin of other biological materials such as prize animals and cultured plants. In an embodiment, reflectance measurements may be combined to determine the location of fiducial points, particularly when using a two-dimensional imager.

During data analysis a simple comparison of spectral measurements with known spectral measurements may be carried out as in known in the art, as for example described in U.S. Pat. No. 6,069,689. However, embodiments of the invention go beyond such simple measurements to obtain more reliable data needed for more demanding assays such as blood glucose measurements. By taking multiple measurements in the same position, spectra at multiple sites, averaging multiple spectra, and/or taking measurements after tensioning a surface, more reliable results may be obtained. These more reliable measurements open a new arena of optical diagnostics that may be carried out non-invasively.

In many embodiments of the invention a probe is applied manually to the sample surface to obtain a measurement. Automated sample surface assay alternately may be used, especially for high value tests such as the selection of successful genetic manipulation of plants or animals. In this context, the invention may be used to solve or alleviate the problem of selecting a tiny number of successful genetic transformations out of a large number of samples based on subtle phenotypic differences that can be determined spectrofluorometrically.

Both automated and manual measurement systems as described here can separate out successful gene transfers. Automated equipment useful for these and other embodiments of the invention are known to skilled artisans. For example, see U.S. Pat. Nos. 5,374,395 (Diagnostics Instrument, 6,162,399 (Universal apparatus for clinical analysis), 6,086,824 (Automatic sample testing machine), 6,025,189 (Apparatus for reading a plurality of biological indicators), 5,955,736 (Reflector assembly for fluorescence detection system), and 5,925,884 (Fluorescence station for biological testing machine), the contents of which relate most closely to automated control systems for which their uses are most particularly included, by reference, as well as the complete disclosures. The materials and methods described in these references can be built into automated fluorescence and/or reflectance instrumentation for embodiments of the invention.

A wide variety of light sources may provide fluorescence excitation and/or a source of light for taking reflectance measurements from the sample. A white light source such as quartz tungston halogen lamp is particularly useful in combination with a light filter such as a glass band pass filter or a grating. Light emitting diodes are particularly useful because of their ability to emit light of a given wavelength range without an optical filter. Presently, and even more so in the future, convenient solid state lasers and other lasers are both commercially available and inexpensive for generating the excitation and/or reflectance light energy origination signal needed. In one embodiment a mechanical shutter or electric switch is used to select between two light sources such as an excitation laser light source or other narrow band source and a white light source. Liquid crystal switches operated by electrical voltage are particularly useful.

A probe according to some embodiments, receives excitation light (or light for reflection) and directs the light to the sample. A preferred probe is an optic fiber bundle, but a skilled artisan will readily appreciate alternative ways to entrain or focus light onto the sample surface in a reproducible manner. A particularly desirable optic fiber bundle is a bifurcated bundle having a merged sampling end wherein fibers from both bundles are mixed to contact the sample surface or are positioned in a defined spatial relationship with the surface. One single end of the bifurcated bundle may direct excitation light (or light for reflection measurements) from a light source into the cable, and the other single end of the bundle may direct emission (or reflected) light from the sample into a detector or imager.

Mixing the two types of fibers allows for a spot on the sample surface to be adjacent to at least one light source fiber and one reflectance/fluorescence light pick up fiber for optic measurements. Other probes may be made, for example from bringing the light source, such as a diode close to the sample. In one embodiment a semiconductor chip is built with a solid state diode laser or non lasing light emitting diode and a detector on the same chip. An array of light emitters and an array of detectors (preferably with light filters as are known in the liquid crystal display thin film transistor art) may be positioned in a pattern on the chip and the chip mounted close to the sample surface as needed. In such cases the light source and/or the light detector may be part of the probe itself.

The probe, in many embodiments directs emitted/reflected light from the sample surface to one or more detectors. A large variety of detectors, both imaging and non-imaging are suitable for various embodiments of the invention. In most instances, a very sensitive photon counting detector may be desirably used. Where photon flux is sufficient and/or gathering optics allow it, less sensitive detection devices, particularly those made from semiconductors, such as charge coupled devices, photo diodes (particularly coupled to low noise high gain amplifiers), and photofets may be used. Preferably an optical filter is interposed between the sample surface and a detector. The optical filter may be a separate unit such as a diffraction grating or an absorption filter or may be part of the probe or detector itself. For example an optical fiber bundle or bundle portion, if used may be constructed from a material that preferentially passes a wavelength region and may act as a filter.

During use a light source typically is turned on and a detector is turned on to operate at the same time. For the detection of fluorescent biological material such as tryptophan or collagen/elastin crosslinks that is particularly useful for glucose detection, an excitation wavelength of about 295 nanometers and an emission wavelength of about 340 nanometers may be preferred. Other wavelengths such as between 200 nanometers and 2400 nanometers are particularly useful as well. In most instances where fluorescence is detected the decay time will be in the nanosecond range and both excitation and emission should take place simultaneously.

The devices and methods also are intended for phosphorescence measurements. For the sake of brevity, the term “fluorescence” as used throughout also includes emissions from longer half-life excited intermediates such as from phosphorescence from molecules, which decay with microseconds or even milliseconds long half life time periods. In some instances the decay time is long enough to allow alternative switching light excitation and emission detection times to improve the signal to noise ratio of the detection step. In such embodiments a light source or shutter is controlled to generate a pulse of light. After the light stops, emission light is collected, to avoid a high background from the excitation light. The materials and methods developed for time resolved fluorescence as, for example described in U.S. Pat. Nos. 5,467,767 (Method for determining if tissue is malignant as opposed to non-malignant using time resolved fluorescence spectroscopy), 5,441,894, (Device containing a light absorbing element for automated chemiluminescent immunoassays), 6,042,785 (Multilabel measurement instrument), and 6,097,025 (Light detection device having an optical path switching mechanism) are particularly useful for this embodiment.

Fluorescence and/or reflectance data obtained by procedures and materials of the invention are analyzed by one or more computational techniques that may be known to skilled artisans. For example, a fluorescence spectral result may be compared with a known standard curve or compared with a reference value that may be pre-set or calibrated into the equipment and used to obtain and analyze a reading. More specifically, a mathematical operation such as dividing one fluorescence signal result with a combined spectra may be carried out to generate a factored spectra. The factored spectra is compared with a stored set of reference factored spectra that have been empirically determined to provide a good decision point. For example, if no greater than 10% variance is acceptable for fluorescence emission between 410 and 460 nanometers is acceptable then if the factored spectra result shows more than 1.1 in this range (measured signal too high) then the fluorescence signal result is deemed “substantially different” and is discarded. Actual mathematical operators, stored set of factors and acceptable variances from the factors may be determined by routinue experimentation.

A wide range of information can be obtained. In preferred embodiments the sample is skin and the fluorescence measurements are used to detect or quantitate both biological states, such as the presence or absence of a specific disease, the progression of a biological phenomenon such as aging, the status of a pre-cancerous condition, and the detection or quantitation of a blood component. Most preferably, blood glucose values are inferred from comparisons between individual spectral measurements, averaged spectral measurements, or from other composite spectral measurements.

### Reduction of Spectral Site to Site Fluorescence Variations

Three ways of reducing spectral site to site variation in fluorescence and/or reflectance signals obtained from a sample surface introduced herein are a) repeated measurements taken at identifiable location(s) determined by fiducial marks, b) measurements repeated at different locations on the sample, and c) tensioning the sample surface during measurement. Combinations of these three ways may be made as desired for each specific application.

1. Repeated measurements via fiducial points or other marks

A problem with repeated measurements, seen in the art previously, is the difficulty in positioning a probe onto the same sample surface for subsequent measurements. In an embodiment this problem is alleviated by providing fiducial points for guidance to determine the bounds of a given sample surface measurement site. The fiducial points are used to more reliably find a sample surface for a repeat measurement.

A chosen surface can be found at least two ways through use of fiducial point(s). In one way, coordinates of the fiducial point(s) allow the user to manually position the probe. For example, a probe can be placed so that a portion touches the sample surface between a series of markings, for more reliable manual placement. In a second way, an imaging device generates a two dimensional image that is operated on by a computer that corrects for small changes in location by determining the same defined sample area between different measurements. That is, the fiducial points inform a computer program as to which constant, defined image region (which in many cases will be near the center of the field) to use for the repeat measurements.

2. Repeated measurements at different locations on a sample

In many cases a sample surface is large enough for multiple readings at different sites and the multiple information obtained is merged to form a more accurate reading compared to measurements taken at a single situs. In one embodiment a probe is placed at successive locations long enough for a stable measurement to be taken at each location. The spectral data is compared and in some instances averaged to form a composite signal. Preferably more than one measurement is taken at each location.

In a preferred embodiment multiple optic readings are taken at each location and one or more of those readings are stored for analysis after that reading has become stable. This embodiment of the invention addresses the problem of taking a measurement when the probe may be shifting position. By looking at successive measurements and only using a measurement after the measured spectrum does not change (or changes less than an arbitrary “acceptable error” value) measurement error from manual placement decreases.

In another embodiment subsequent measurements are taken at different locations that may not overlap with locations used for earlier measurements. For example, measurements may be taken at 2 or more, more preferably 3 or more, still preferably 5 or more and even more preferably at 10 or more locations. In an embodiment the center of each probe location is at least 1 mm away from locations used for previous measurements, yet more preferably is at least 2 mm away, and may be at 5 mm distant, or even more than 10 mm distant, depending on other factors such as the homogeneity of the sample surface.

To facilitate rapid acquisition of data by feedback to the user, an instrument according to an embodiment of the invention may monitor the optic signal continuously and determine when the signal is stabile (indicating a non-moving probe on the sample surface). When the signal stops changing the electronic fluorescence signal is input into a data analyzer and optionally the unit alerts the user to move the probe to a new location by an audible beep or other indication.

3. Measurements from a tensioned surface

The inventors discovered that samples with some elasticity such as skin could be tensioned during the spectral measurement and thereby provide more reliable data. In preferred embodiments tensioning occurs mechanically.

In one embodiment, four fiducial points located at opposite corners from a center spot for taking a spectroscopic measurement are spread apart by 0.1% to 1% (measured with respect to the diagonal between opposite points, running through the center of the four points) through friction fitting or mechanical coupling of a probe. In another embodiment the points are spread apart by 1 to 5%, and in another embodiment the points are spread apart by more than 5%. In yet another embodiment the points are spread apart by more than 10% and in yet another embodiment the points are spread apart by more than 20%. These relative degrees of spreading (1%, 2%, 5%, 10% and 25%) are dimensionless and are herein termed “tension values.” A tension value in practice can be measured in any preferred units and spacing depending on the actual sample surface being tested.

The fiducial point(s) or mark(s) may be in the form of two or more dots or other shapes that adhere to the sample surface. Adhesive agents such as glues, tapes, magnetic clamps, pinchers, suction devices, pins, nails, and the like are known and are contemplated for embodiments of the invention. In practice, two or more and preferably at least 4 points are affixed to the sample. A probe, or probe holder having complementary attachments to the fiducial points is attached. In most embodiments complementary attachments on the probe or probe holder are positioned slightly further apart such as between 0.1 to 1%, 1% to 5% or more than 5% apart (measured with respect to the diagonal between opposite points, running through the center of the four points). Attaching the probe or probe holder to the fiducial points thus causes spreading of the sample surface by the amount of mismatch between the fiducial points and their matching connect points to the probe or probe holder.

A wide variety of shapes and sizes of fiducial points are useful. The term “fiducial points” has been used for convenience, but embodiments of the invention utilize other attachment types that depart from a point shape. For example, an elastic ring can be affixed to the skin, having an inner area that is slightly smaller than the body of the probe. A matching end of the probe can, for example, be friction inserted into the ring, causing the elastic ring and the sample surface to spread apart from their centers. This spreading can decrease folds and wrinkles within the ring. As used herein the term “fiducial points” refers to multiple attachments to a sample surface that can mechanically couple to a probe or probe holder. A ring has very many attachments, while other shapes or even points are useful as long as the surface attachments are made in two dimensions (i.e. not limited to a single line only, as between two points only).

In another embodiment of the invention, a sample surface such as skin is tensioned by contacting an enclosed volume and applying a vacuum within within the enclosed volume, thus pulling a portion of the skin outside of its normal two dimensional plane onto or near a spectroscopic probe surface or opening. The vacuum conveniently can be formed manually by operation of a flexible diaphragm that alters the confined volume. In one embodiment skin is pulled into the volume due to the lower pressure and contacts one or more surfaces of the probe end to form a more reproducible optical target of the probe. In another embodiment the vacuum tensioned sample contacts a positioning reference surface such as a plastic bar, frame or other stop, and the probe takes a measurement with a known positioning or spacing between sample surface and probe. In yet another embodiment one or parts of the device that contact the sample surface are disposable and comprise, for example, paper, plastic or other material that may be manufactured inexpensively.

### EXAMPLES

### Example 1

This example demonstrates the use of fiducial points in making repeated measurements and tensioning a sample surface for improved fluorescence data measurements.

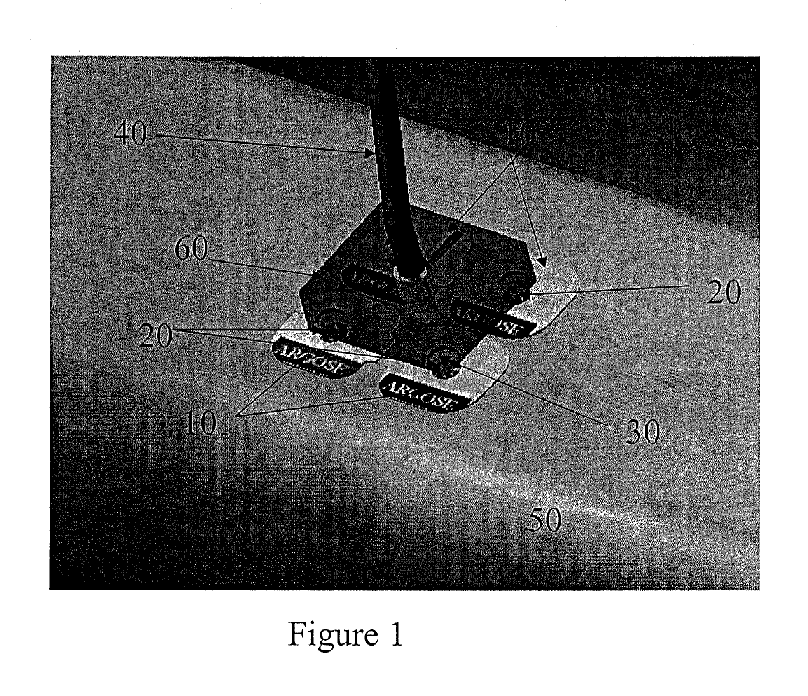

FIG. 1 shows four adhesive pads 10, which contain an adhesive for binding to skin surface 50 on their lower surfaces. Each pad 10 contains a fiducial point 20 attached to its center. Receiver 60 holds fiber optic probe 40 and contains four mating dimples 30, which correspond to and form a mechanical connection with fiducial points 20. Only one dimple shown has an associated arrow in the figure for clarity. The dimples allow repeated positioning of receiver 60, and hence fiber optic 40, which is connected to receiver 60.

The inter-dimple spacing for dimples 30 is approximately 3% greater than the spacing between fiducial points 20. Upon application of receiver 60 through formation of mechanical contacts between dimples 30 and points 20 skin surface 50 that attached to fiducial points 20 is tensioned. In a preferred embodiment pads 10 are EKG pads and fiducial points 20 are male snaps that mate with the EKG pads and to which EKG electrodes normally are attached. In this arrangement the dimples are female snaps.

After connecting receiver 60, an instrument that generates a 395 wavelength maximum excitation light and records fluorescence emission is attached to the distal end of fiber optic 40 (not shown) and measurements are taken. Blood glucose measurements obtained by tensioning the skin sample 3% are found to be more accurate than glucose measurements obtained without tensioning.

### Example 2

This example demonstrates the use of simultaneous multiple sample measurements with a single probe. The probe contacts a sample surface and four measurements are made at four independent locations on the surface.

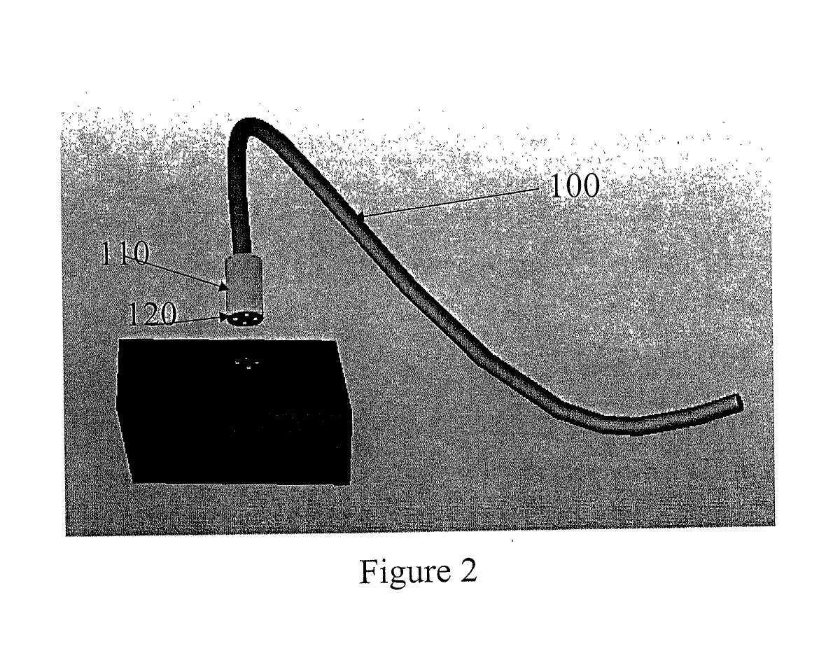

FIG. 2 shows fiber optic probe 100 having four apertures 120 that are spaced within ferrule 110. Apertures 120 are spaced 10 mm apart (center to center measurements). The complete fiber optic probe 100 contain 64 fibers. Each fiber is 200 micrometers in diameter and each of the four apertures 120 contains 16 of the fibers. This arrangement is used to sample four skin tissue sites simultaneously at a distance such that each aperture records an optic signal from an independent sample as described in Example 1.

### Example 3

This example demonstrates imaging of fluorescence spectra from four samples with a single probe simultaneously.

In this example, the cross sectional ordering of fibers in each aperture as shown in FIG. 2 are maintained. The blood glucose concentration of a person is measured fluorometrically as described in Example 2 except that spectra from each of the four sites are measured simultaneously but distinguishably by an imaging spectrometer. In this latter embodiment, the spectra are analysed by a computer that accepts data from the imaging spectrometer. During this analysis, the spectra are examined for outliers, and non-representative spectra discarded. It is found that use of imaging provides blood glucose concentration measurements that are more precise than measurements obtained with a non imaging method.

### Example 4

This example demonstrates the use of fiducial points for improved precision of fluorescence measurements from skin.

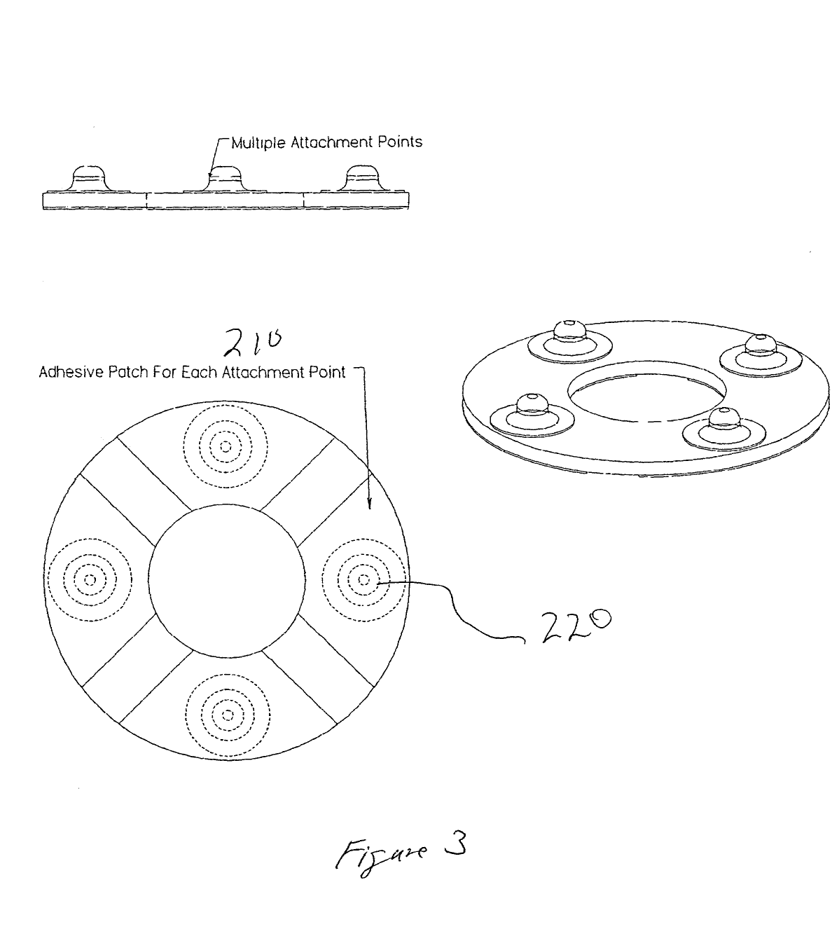

FIG. 3 shows a one piece mounting surface with multiple attachment points which provide positional repeatability for applying a fluorescence spectral probe to the skin. In this example, adhesive patches 210 form contact surfaces that allow independent movement of individual mounting points 220. When more movement is required than the mounting material allows, the separate sections can move.

In another example (not shown) the fiber optic probe 40 of FIG. 1 contains a ferrule with multiple apertures as exemplified by ferrule 110 and apertures 120 in FIG. 2.

Other embodiments of the invention will be apparent to those skilled in the art from consideration of the specification and practice of the invention disclosed herein. All references cited herein, including all U.S. and foreign patents and patent applications, are specifically and entirely incorporated by reference. It is intended that the specification and examples be considered exemplary only, with the true scope and spirit of the invention indicated by the following claims.

1. A method of minimizing error in optic spectra from a sample comprising the steps of:
applying one or more fixed fiducial points to the sample surface; and
referencing an optical probe to said one or more fiducial points, so that the spectra are taken in the same place.

2. The method of claim 1, wherein at least 1 fiducial point is applied to the sample surface.

3. The method of claim 1, wherein at least 2 fiducial points are applied to the sample surface.

4. The method of claim 1, wherein at least 3 fiducial points are applied to the sample surface.

5. The method of claim 1, wherein the sample surface is skin of a living body.

6. The method of claim 4, wherein the fluorescence spectra information is used to determine the level of an analyte in the body.

7. The method of claim 5 in which the analyte is glucose.

8. The method of claim 1, wherein the optical probe comprises a fiber optic bundle.

9. The method of claim 8, wherein the optic bundle is bifurcated and contains at least 16 light conducting fibers.

10. The method of claim 1, wherein a plurality of spectra are combined to form a representative spectrum by the further steps:
comparing a spectra measurement with a combined spectra to generate a compared spectra;
discarding the compared spectra if substantially different from a reference; and
combining the remaining spectra to form a representative spectrum.

11. A method of minimizing the variation of optic spectra from a sample comprising the steps of:
gathering a plurality of spectra at nearby points on the sample; and
combining the spectra so as to form a representative measurement.

12. The method of claim 11, wherein the sample surface is skin of a living body.

13. The method of claim 12, wherein the fluorescence spectra information is used to determine the level of an analyte in the body.

14. The method of claim 13 in which the analyte is glucose.

15. The method of claim 11, wherein the optical probe comprises a fiber optic bundle.

16. The method of claim 15, wherein the optic bundle is bifurcated and contains at least 16 light conducting fibers.

17. The method of claim 15, wherein the optic bundle contains at least 64 light conducting fibers.

18. The method of claim 11, wherein the probe contains at least two apertures, each of which acquires a fluorescence measurement at a different location on the sample.

19. The method of claim 18, wherein a plurality of spectra are combined to form a representative spectrum by the further steps:
a) comparing a spectra measurement with a combined spectra to generate a compared spectra;
b) discarding the compared spectra if substantially different from a reference; and
c) combining the remaining spectra to form a representative spectrum.

20. A method of minimizing the variation of measured optic spectra from a flexible sample surface comprising tensioning the sample surface prior to or at the time of making a spectral measurement with an optical probe.

21. The method of claim 20, wherein tensioning is carried out by: adhering one or more fiduciary marks on the skin to provide a friction fitting
a) contact with the probe;
b) inserting the probe into the friction fitting contact;
c) making a spectral measurement from the probe; and
d) repeating steps b) and c) for successive measurements.

22. The method of claim 20, wherein the sample surface is skin of a living body.

23. The method of claim 21, wherein the fluorescence spectra information is used to determine the level of an analyte in the body.

24. The method of claim 22 in which the analyte is glucose.

25. The method of claim 20, wherein the optical probe comprises a fiber optic bundle.

26. The method of claim 25, wherein the optic bundle is bifurcated and contains at least 16 light conducting fibers.

27. The method of claim 26, wherein the optic bundle contains at least 64 light conducting fibers.

28. The method of claim 20, wherein the probe contains at least two apertures, each of which acquires a fluorescence measurement at a different location on the sample.

29. The method of claim 28, wherein a plurality of spectra are combined to form a representative spectrum by the further steps:
c) comparing a spectra measurement with a combined spectra to generate a compared spectra;
d) discarding the compared spectra if substantially different from a reference; and
e) combining the remaining spectra to form a representative spectrum.
